(19) United States
(12) Patent Application Publication
(10) Pub. No.: US 2016/0157543 A1
(43) Pub. Date: Jun. 9, 2016

(54) DEVICE TO REDUCE TRAUMATIC BRAIN INJURY

(71) Applicant: Chiming Huang, Shawnee Mission, KS (US)

(72) Inventor: Chiming Huang, Shawnee Mission, KS (US)

(73) Assignee: Chiming Huang, Shawnee Mission, KS (US)

(21) Appl. No.: 14/959,083

(22) Filed: Dec. 4, 2015

Related U.S. Application Data
(60) Provisional application No. 62/088,181, filed on Dec. 5, 2014.

(51) Int. Cl. A42B 3/04; A63B 71/10; A61F 5/37; A61B 5/11; A61B 5/00

(52) U.S. Cl. CPC A42B 3/0433; A42B 3/0473; A61B 5/11; A61B 5/7282; A61F 5/3707; A63B 71/10

(57) ABSTRACT

A device for reducing traumatic brain injury comprises a first sensor, a first linkage element, and a processing element. The first sensor is coupled to a head component and configured to measure an acceleration of a user's head and to generate a sequence of real-time measured samples. The first linkage element is configured to connect the head component to a body component and is able to switch between a first state in which it is relatively flexible and a second state in which it is relatively rigid. The first linkage element is switched from its first state to its second state by a locking signal. The processing element is configured to receive the real-time measured samples and to generate the locking signal when each of a portion of the real-time measured samples is greater than one of a corresponding portion of a plurality of dynamic concussion thresholds.

### RELATED APPLICATIONS

The current patent application is a non-provisional patent application which claims priority benefit with regard to all common subject matter to U.S. Provisional Patent Application Ser. No. 62/088,181, titled “DEVICE, SYSTEM, AND METHOD TO REDUCE TRAUMATIC BRAIN INJURY: THE SENSOR STAGE”, filed Dec. 5, 2014. The provisional application is hereby incorporated by reference in its entirety into the current patent application.

### BACKGROUND OF THE INVENTION

1. Field of the Invention

Embodiments of the current invention relate to devices configured to reduce traumatic brain injury.

2. Description of the Related Art

Closed-head traumatic brain injury (TBI) is typically a result of the brain impacting the interior of the skull. Forces acting on the body or the head generally accelerate the brain. High positive acceleration or negative acceleration may cause the brain to contact the skull with enough force to cause brain injury. The types of brain injury may be categorized as blast TBI, concussive TBI, or mild TBI, etc. Blast TBI may be experienced by military or law enforcement personnel while on patrol or traveling in a vehicle. Concussive TBI may be suffered by athletes in sports such as hockey, boxing, or American football. Mild TBI may be experienced by anyone suffering a fall, a vehicular accident, or the like.

Systems that have been developed for preventing concussions for players of American football may include one or more acceleration sensors coupled to a football helmet and one or more mechanisms connecting the helmet to shoulder pads. The systems may further include a processing element that locks the connecting mechanisms when the acceleration measured by the sensors exceeds a certain constant value believed to be a threshold beyond which a concussion to the player may occur. At least one drawback to this approach is that the threshold for concussion may be different for different people. As an example, an NFL player may endure a hit with an acceleration of 30-60 G (acceleration due to gravity) without injury, whereas the same hit may cause a concussion or other serious injuries in a little league football player. Furthermore, the threshold for an individual may change over time or be dependent upon other factors, all of which render it impractical to adopt a certain, one-size-fits-all constant value as a threshold beyond which a concussion is expected to occur.

Another drawback to the current approach is that sensors in many prior arts are programmed to sample the peak amplitudes of the impact force/energy, typically requiring ˜100 ms to manifest. In boxing matches, a concussion in the form of a knockout may take place within ˜100 ms after impact. The current approach of sensing impact force/energy may be therefore too slow to be incorporated in a device designed for launching an effective countermeasure for the purpose of preventing a concussion.

### SUMMARY OF THE INVENTION

Embodiments of the current invention solve the above-mentioned problems and provide methods and devices that are utilized with head gear and body wear to reduce traumatic brain injury.

A first embodiment of the current invention provides a device for reducing traumatic brain injury and broadly comprises a first sensor, a first linkage element, and a processing element. The first sensor is coupled to a head component and configured to measure an acceleration of a user's head as a result of motion of the head component and to generate a sequence of real-time measured samples. The first linkage element is configured to connect the head component to a body component and is able to switch between a first state in which it is relatively flexible and a second state in which it is relatively rigid so that an impedance-preferred pathway is established for impact energy dissipation away from the head and toward the body of the user. The first linkage element is switched from its first state to its second state by a locking signal which is generated by a processing element after the impact but before the impact energy can cause a concussion. The processing element is configured to receive the real-time measured samples on head motion from the first sensor and to generate the locking signal when each of a portion of the real-time measured samples is greater than one of a corresponding portion of a plurality of dynamic concussion thresholds.

A second embodiment of the current invention provides a system for reducing traumatic brain injury and comprises a first sensor and a second sensor, a first linkage element, and a processing element. The first sensor is coupled to a head component and configured to measure an acceleration of a user's head as a result of motion of the head component and to generate a sequence of real-time measured samples. The second sensor is coupled to a body component and configured to measure an acceleration of a user's body as a result of motion of the body component and to generate a sequence of real-time measured samples. The first linkage element is configured to connect the head component to a body component and is able to switch between a first state in which it is relatively flexible and a second state in which it is relatively rigid so that an impedance-preferred pathway is established for impact energy dissipation away from the head and toward the body of the user. The first linkage element is switched from its first state to its second state by a locking signal which is generated by the processing element after the impact but before the impact energy can cause brain injury. The processing element is configured to receive the real-time measured samples from the first sensor and the second sensor and determine a period of motion which includes the real-time measured samples whose value is greater than a motion threshold. The processing element may determine a profile on normal, voluntary, non-injurious, and non-concussion-inducing movement of the head and the body of the individual user based on a plurality of biomechanical parameters (head velocities, accelerations, etc.). The processing element may further compute sequential dynamic concussion thresholds derived from the profile on normal, voluntary, non-injurious, and non-concussion-inducing movement of the head and the body of the individual user, and compare, in sequential order, each of the real-time measured samples in the period of motion with the corresponding dynamic concussion thresholds.

A third embodiment of the current invention provides a method of reducing traumatic brain injury comprising the steps of: receiving a sequence of real-time measured samples from a first sensor coupled to a head component, determining a period of motion which includes the real-time measured samples whose value is greater than a motion threshold, calculating a dynamic concussion threshold for each of a plurality of sequential time-based profile cells, comparing, in sequential order, each of the real-time measured samples in the period of motion with the corresponding dynamic concussion threshold, and generating a locking signal if necessary.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the current invention will be apparent from the following detailed description of the embodiments and the accompanying drawing figures.

### BRIEF DESCRIPTION OF THE DRAWING FIGURES

Embodiments of the current invention are described in detail below with reference to the attached drawing figures, wherein:

FIG. 1 is a perspective view of a device, seen from the rear, for reducing traumatic brain injury constructed in accordance with a first embodiment of the current invention and utilized with an American football helmet and shoulder pads, the device including first and second sensors and first and second linkage elements;

FIG. 2 is a rear elevational view of the device of FIG. 1;

FIG. 3a is a sectional view of a first linkage element, the first linkage element including a first member, a second member, a bypass element, and a fluid medium, wherein FIG. 3a depicts the second member telescoping in to the first member and moving the fluid medium in a first direction;

FIG. 3b is a sectional view of the first linkage element, wherein FIG. 3b depicts the second member telescoping out of the first member and moving the fluid medium in a second direction, opposite of the first direction;

FIG. 4 is a sectional view of the first linkage element, the first linkage element further including a locking element;

FIG. 5 is a schematic block diagram of at least some of the electronic components of the device of FIG. 1;

FIG. 6 is a block flow diagram depicting at least a portion of the creation of a profile for use with the device of FIG. 1;

FIG. 7 is a block flow diagram depicting at least a portion of the comparison of real-time measured samples with dynamic concussion thresholds for use with the device of FIG. 1;

FIG. 8 is a sectional view of a second embodiment of the first linkage element;

FIG. 9 is a sectional view of a third embodiment of the first linkage element;

FIG. 10 is a sectional view of a fourth embodiment of the first linkage element;

FIG. 11 is a flow diagram of at least a portion of the steps of a method for creating a profile to be used with a device for reducing traumatic brain injury in accordance with another embodiment of the current invention; and

FIG. 12 is a flow diagram of at least a portion of the steps of a method for reducing traumatic brain injury in accordance with yet another embodiment of the current invention.

The drawing figures do not limit the current invention to the specific embodiments disclosed and described herein. The drawings are not necessarily to scale, emphasis instead being placed upon clearly illustrating the principles of the invention.

### DETAILED DESCRIPTION OF THE EMBODIMENTS

The following detailed description of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the present invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the current technology can include a variety of combinations and/or integrations of the embodiments described herein.

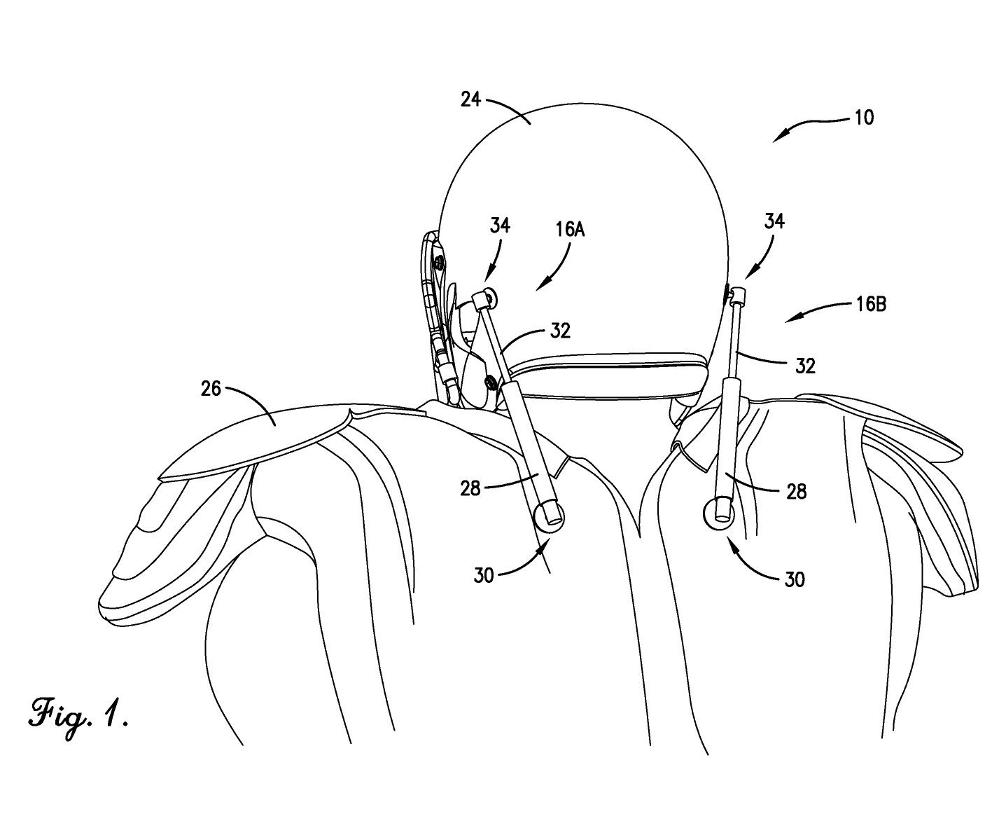

A device 10 for reducing traumatic brain injury constructed in accordance with a first embodiment of the current invention is shown in FIGS. 1, 2, and 5 and broadly comprises a first sensor 12, a second sensor 14, a first linkage element 16A, a second linkage element 16B, a communication element 18, a memory element 20, and a processing element 22. The device 10 may be utilized by a user engaging in activity during which a concussion may possibly be caused by an impact to the head or to the body. The activity may include contact sports such as hockey, boxing, American football, soccer, snow or ice-related sports such as skiing, snowboarding, sledding, sports in which falling or landing on the head is possible such as skateboarding, bicycling, equestrian activities, motorcycle riding, automobile driving, also including military combat, and the like. In a typical usage scenario for the current invention, the device 10 may interface with equipment worn on the head or the body including a head component 24, such as a helmet or other headgear, and a body component 26, such as the shoulder pads in American football or the body armor worn by soldiers in combat.

The first sensor 12, indicated in FIG. 5, generally measures linear as well as rotational motion of the user's head. The term “sensor” may include a plurality of sensors or types of sensors. Thus, the first sensor 12 may include motion sensors, velocity sensors, shock sensors, accelerometers, gyroscope chips, magnetometer chips, inclinometers, angle rate sensors, angular velocity sensors, vibration sensors, or the like, or combinations thereof. The first sensor 12 may include technology such as strain gauges, piezoelectric elements, micro electro-mechanical systems (MEMS), nanotechnologies in which a material, solid or liquid, can change its stiffness while modulated by electromagnetic fields, or the like, or combinations thereof. The first sensor 12 may measure linear position, velocity, acceleration, or force along a single axis or multiple axes, such as any three mutually orthogonal axes, e.g., the X, Y, Z axes, and may record, communicate, or output a sensor measurement. Each sensor measurement may include a plurality of values from a plurality of sensors which may be in the form of vector data or magnitude data. Thus, in various embodiments, the first sensor 12 may generate three or more values for the three linear measurements. In addition or instead, the first sensor 12 may measure angular or rotational position, velocity, acceleration, or force concerning head movement along mutually orthogonal axes, such as pitch, roll, and yaw. The second sensor 14 may measure angular or rotational position, velocity, acceleration, or force concerning body movements along mutually orthogonal axes, such as front-to-back, left-to-right, and up-and-down. With regard to measuring the acceleration of the head, pitch is nodding to gesture yes, roll is bending the head-and-neck toward one or the other shoulder, and yaw is gesturing no or turning the head to watch cars from both directions before crossing a street. Accordingly, the first sensor 12 may generate three or more values for the three angular measurements.

The sensor measurements may be an analog value, a digital value, a pulse-width modulation (PWM) value, or the like. An exemplary first sensor 12 may output the sensor measurements as real-time measured samples at an exemplary frequency ranging from 500 hertz (Hz) to 20 kilohertz (kHz) or higher. This range of frequencies should be great enough to detect an impulse-like impact, whose duration may be range from a fraction of a millisecond to single and up to double digits of milliseconds. The first sensor 12 may also include electronic circuitry such as amplifiers, analog-to-digital converters (ADCs), or other conversion circuits.

The first sensor 12 may be positioned within the interior of the head component 24. The head component 24 may be headwear, headgear, a helmet, such as a sports helmet, a motorcycle or automobile helmet, or a combat helmet, or the like. In some embodiments, the first sensor 12 may further include first and second resilient members, such as springs, that are coupled to opposing sides of the first sensor 12. The first resilient member may contact an inner surface of the head component 24, and the second resilient member may contact the user's head. In other embodiments, the first sensor 12, with or without resilient members, may be coupled to padding on the interior of the head component 24, or coupled to a hard shell of the head component 24, such that when the head component 24 is worn, the first sensor 12 may contact the user's head in order to detect motion, force, and other physical parameters related to the force applied to the head or the helmet. It may be advantageous for the first sensor 12 to also measure the force at the helmet, which is typically of a greater magnitude than the force at the head.

The second sensor 14, indicated in FIG. 5, may be substantially similar to the first sensor 12 in structure and function. The second sensor 14 may be positioned on the body component 26 and may measure motion of the user's body. Thus, the second sensor 14 may generate data based on the motion of the user's body, particularly the user's upper body (as oppose to limb movements).

The first linkage element 16, as seen in FIGS. 1-4, generally provides a link between the user's head and the user's body that is normally flexible but becomes rigid upon receiving a locking signal from processing element 22 as a result of an impact to the head or the body that could potentially cause a concussion. The first linkage element 16 may include a first member 28, a first end joint 30, a second member 32, a second end joint 34, a bypass element 36, and a locking element 38.

The first member 28 may be of generally hollow, cylindrical or tubular construction and may include a chamber defined by a circumferential sidewall 40, a first end wall 42 connected to one end thereof, and a second end wall 44 connected to the opposing end thereof. The first end wall 42 may be generally disc shaped. The second end wall 44 may be generally disc shaped with a circular opening in the center thereof. The first member 28 may further include a fluid medium 46 housed within the chamber. The fluid medium 46 may include liquids, such as hydraulic fluids (including water), or gases, such as atmospheric air.

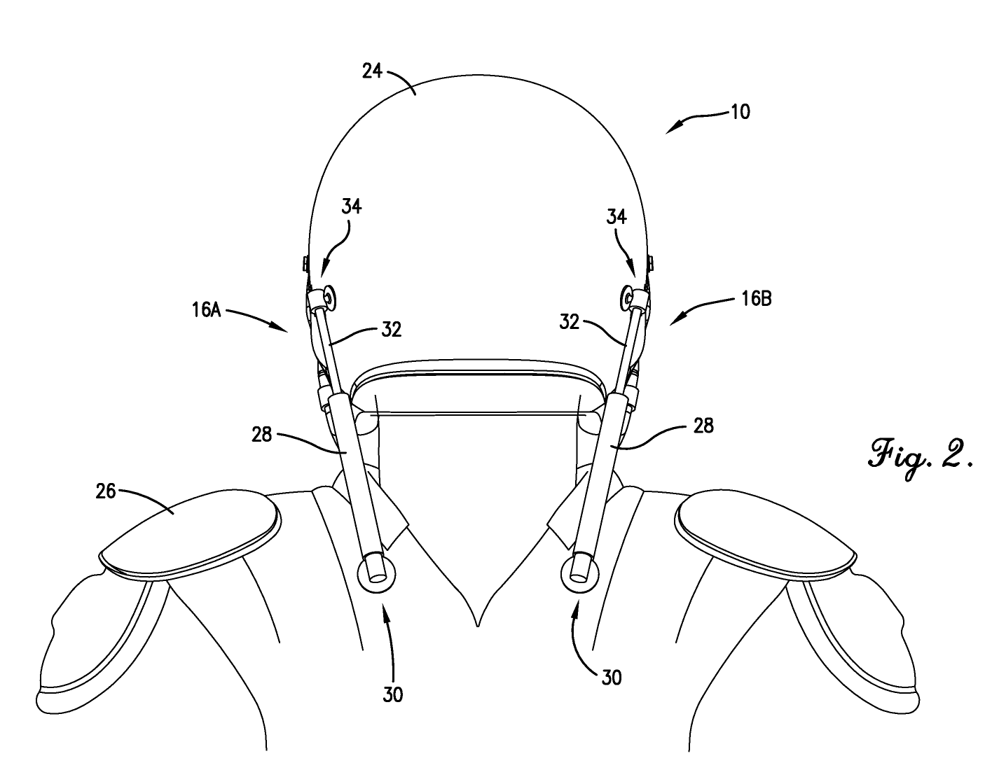

The first end joint 30, seen in FIGS. 1 and 2, may connect the first member 28 to the body component 26 and may include a universal joint with a first connector coupled to the sidewall 40 or the first end wall 42 of the first member 28 and a second connector coupled to the body component 26 in a location corresponding to the left side of the upper back of the user. The universal joint may provide rotation about three orthogonal axes such that the first member 28 may freely rotate in any direction with respect to the body component 26.

The second member 32 may include a plunger with an elongated rod 48 and a disc 50 connected to one end thereof. The disc 50 may be positioned within the chamber of the first member 28, and the rod 48 may extend through the opening in the second end wall 44 of the first member 28. During operation of the device 10, discussed in more detail below, the second member 32 may travel or slide axially within the first member 28 in a telescoping fashion. Both the rod 48 and the disc 50 may contact and interact with the fluid medium 46.

The second end joint 34, seen in FIGS. 1 and 2, may be similar in structure and function to the first end joint 30. The second end joint 34 may include a first connector coupled to the rod 48 of the second member 32 and a second connector coupled to the head component 24 on one side thereof, corresponding to the left side of the user's head. The second end joint 34 may provide rotation about three orthogonal axes such that the second member 32 may freely rotate in any direction with respect to the head component 24.

The bypass element 36 generally provides a path for the fluid medium 46 of the first member 28 to recirculate. The bypass element 36 may include a chamber, a first port positioned at one end of the chamber, and a second port positioned at an opposing end of the chamber. The first and second ports may each provide fluid access to the chamber. The first port may be coupled to the first member 28 at, or near, the first end wall 42, while the second port may be coupled to the first member 28 at, or near, the second end wall 44. In some embodiments, the bypass element 36 may be positioned adjacent to the first member 28. In other embodiments, the bypass element 36 may have a cylindrical shape that forms a chamber surrounding the first member 28.

When the rod 48 of the second member 32 is free to travel or slide axially with respect to the first member 28, the linkage element 16 may change its length, thereby allowing the head component 24 to move freely relative to the body component 26. Under this condition, the linkage element 16 is said to be in a flexible state.

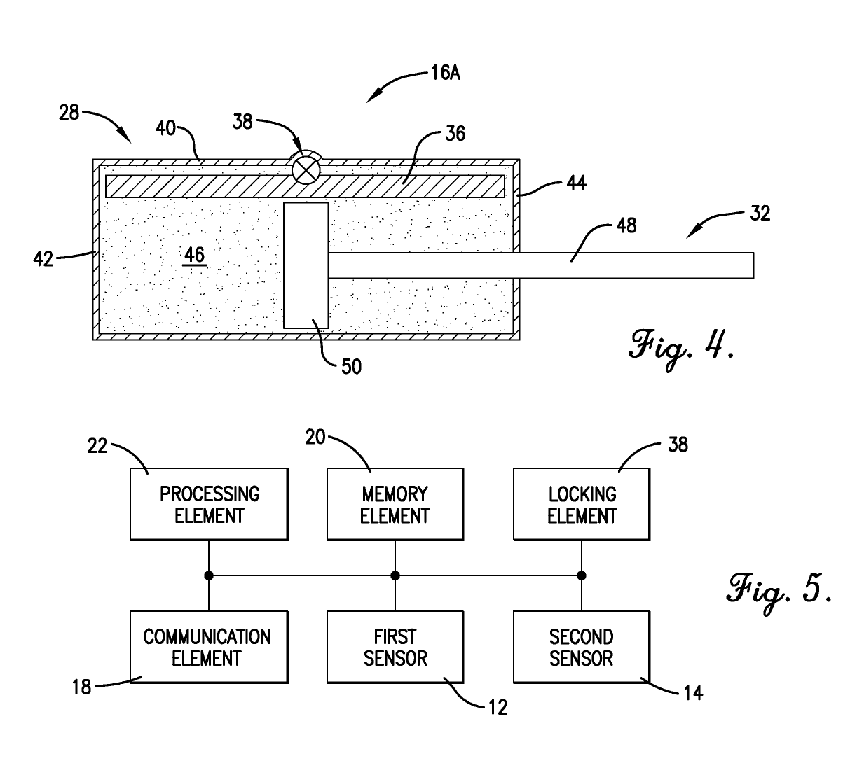

The locking element 38, seen in FIG. 4, generally is capable to change the state of the linkage element 16 from flexible to rigid and may include devices, such as solenoid valves with solenoids that deliver a mechanical action when provided with an electronic signal. The locking element 38 may be coupled to the bypass element 36 and may function as a valve which stops the flow of the fluid medium 46 through the bypass element 36 when it receives a locking signal from the processing element 22. The locking element 38 may include components that interact or function with hydraulic or pneumatic systems. In an exemplary embodiment, the locking element 38 may include a solenoid valve with a movable core or plunger that can be extended from and retracted into a body. The solenoid valve may be attached to the bypass element 36 in series such that the solenoid valve may stop the flow of the fluid medium 46 by ejecting a core element within the chamber of the solenoid valve and into the flowing path of the fluid medium 46. In other words, when the locking element 38 receives the locking signal, the core of a solenoid valve may be ejected into the flowing path of fluid medium 46 within the chamber of the bypass element 36 such that it completely blocks the path of the fluid medium 46, stopping the flow thereof.

When the flow of the fluid medium 46 is stopped, the rod 48 of the second member 32 is no longer free to travel or slide axially with respect to the first member 28, the linkage element 16 may not change its length, thereby preventing the head component 24 from moving freely relative to the body component 26. Under this condition, the linkage element 16 is said to be in a rigid state.

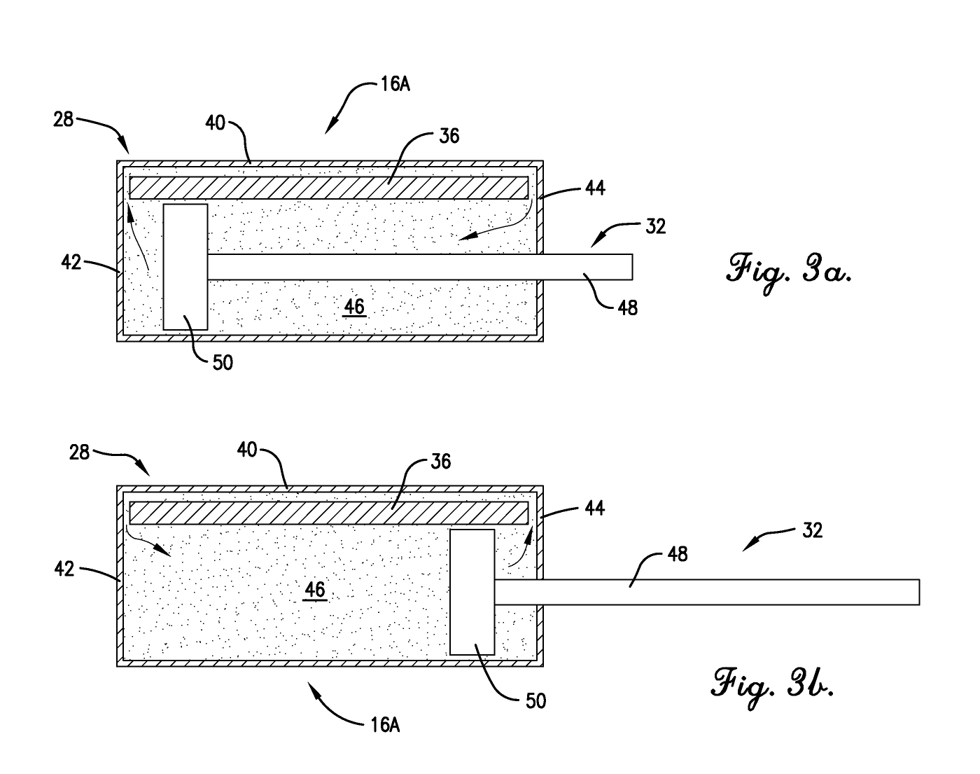

The first linkage element 16 may function as follows. In the flexible state, given the connection of the first linkage element 16 to the head component 24 and the body component 26, every time the user moves his head the second member 32 may telescope, or piston, in and out of the first member 28. While the second member 32 is moving inward as seen in FIG. 3a, the disc 50 may push the fluid medium 46 toward the first end wall 42. The fluid medium 46 may exit the first member 28 through the first port of the bypass element 36, travel through the chamber, and enter the first member 28 again through the second port of the bypass element 36 proximal to the second end wall 44. While the second member 32 is moving outward as seen in FIG. 3b, the process may reverse, with the fluid medium 46 flowing in the opposite direction, such that the disc 50 may push the fluid medium 46 toward the second end wall 44. The fluid medium 46 may exit the first member 28 through the second port of the bypass element 36, travel through the chamber, and enter the first member 28 again through the first port of the bypass element 36 proximal to the first end wall 42. As long as the locking element 38 is not activated or energized, the fluid medium 46 may flow freely through the chambers of the first member 28 and the bypass element 36. In the flexible state, therefore, the length of the linkage element 16 is free to change and the head element 24 is free to move relative to the body element 26.

In the rigid state, the locking element 38 is energized, preventing the flow of the fluid medium 46 through both the chamber of the first member 28 and the chamber of the bypass element 36. When the fluid medium 46 does not flow, the second member 32 may not move with respect to the first member 28—effectively locking or holding constant the length of the linkage element 16. In the rigid state, therefore, the length of the linkage element 16 (i.e. 16A, 16B) is fixed and not free to change, the head element 24 is not free to move relative to the body element 26.

The second linkage element 16B, as seen in FIGS. 1 and 2, may be substantially similar to the first linkage element 16A in structure and function and may include the same components discussed above. The first end joint 30 of the second linkage element 16B may be coupled to the body component 26 in a location corresponding to the right side of the upper back of the user. The second end joint 34 of the second linkage element 16B may be coupled to the head component 24 on one side thereof, corresponding to the right side of the user's head. The second linkage element 16B may operate in combination with the first linkage element 16A to prevent movement of the head with respect to the body when a potentially injurious impact to the head or body is detected.

To summarize the description up to this point, the linkage elements 16A and 16B may change from a flexible state that does allow head movements to a rigid state that does not allow head movements. In the rigid state, linkage element 16A and 16B provide an inflexible and impedance-preferred pathway for the efficient dissipation of impact energy. Therefore, when the linkage elements 16A and 16B are in the rigid state, the impact energy is channeled and dissipated to the trunk or the body and is not able to cause brain injury. This mechanism may be provided by fast technology that may involve miniaturized solenoid valves and the like. The response time of such valves are generally in single digit of milliseconds (ms).

The communication element 18, indicated in FIG. 5, generally allows communication with external systems or devices. The communication element 18 may include signal or data transmitting and receiving circuits, such as antennas, amplifiers, filters, mixers, oscillators, digital signal processors (DSPs), and the like. The communication element 18 may establish wireless communication by utilizing radio frequency (RF) signals and/or data that comply with communication standards such as cellular 2G, 3G, or 4G, Institute of Electrical and Electronics Engineers (IEEE) 802.11 standard such as WiFi, IEEE 802.16 standard such as WiMAX, Bluetooth™, or combinations thereof. In addition, the communication element 18 may utilize communication standards such as ANT, ANT+, Bluetooth™ low energy (BLE), the industrial, scientific, and medical (ISM) band at 2.4 gigahertz (GHz), or the like. Alternatively, or in addition, the communication element 18 may establish communication through connectors or couplers that receive metal conductor wires or cables which are compatible with networking technologies such as ethernet. In certain embodiments, the communication element 18 may also couple with optical fiber cables. The communication element 18 may be in communication with the processing element 22 and the memory element 20.

The memory element 20, indicated in FIG. 5, may include electronic hardware data storage components such as read-only memory (ROM), programmable ROM, erasable programmable ROM, random-access memory (RAM) such as static RAM (SRAM) or dynamic RAM (DRAM), cache memory, hard disks, floppy disks, optical disks, flash memory, thumb drives, universal serial bus (USB) drives, or the like, or combinations thereof. In some embodiments, the memory element 20 may be embedded in, or packaged in the same package as, the processing element 22. The memory element 20 may include, or may constitute, a “computer-readable medium”. The memory element 20 may store the instructions, code, code segments, software, firmware, programs, applications, apps, services, daemons, or the like that are executed by the processing element 22. The memory element 20 may also store settings, data, documents, sound files, photographs, movies, images, databases, and the like.

The processing element 22, indicated in FIG. 5, may include electronic hardware components such as processors, microprocessors (single-core and multi-core), microcontrollers, digital signal processors (DSPs), field-programmable gate arrays (FPGAs), analog and/or digital application-specific integrated circuits (ASICs), or the like, or combinations thereof. The processing element 22 may generally execute, process, or run instructions, code, code segments, software, firmware, programs, applications, apps, processes, services, daemons, or the like. The processing element 22 may also include hardware components such as finite-state machines, sequential and combinational logic, and other electronic circuits that can perform the functions necessary for the operation of the current invention. The processing element 22 may be in communication with the other electronic components through serial or parallel links that include address busses, data busses, control lines, and the like.

The processing element 22 may receive data from the first sensor 12 and the second sensor 14 on the user's head movement or body movement. The data may include measurements of position or distance, velocity, and acceleration. Alternatively, these parameters may be derived from the first and second sensors 12 and 14. The processing element 22 may be operable to, configured to, or programmed to utilize the data to generate a profile of one or more of the above-mentioned parameters on the user's head movement or body movement as described below. In various embodiments, the processing element 22 may be programmed to generate a profile for each one of the parameters, resulting in a position profile, a velocity profile, and an acceleration profile, and so on. In addition, there may be a profile for each parameter from each of the first and second sensors 12, 14. These profiles may offer a description on the range, the mean value, and the variation concerning the user's normal, voluntary, non-injurious, and non-concussion-inducing head movement or body movement. Each profile may further include a plurality of sequential time-based cells for the given parameter, wherein each cell includes information on the statistics from data collected over an extended period. Such information constitutes a detailed and historical profile of the personal data on the user's normal, voluntary, non-injurious, and non-concussion-inducing head movement or body movement collected over an extend period, e.g. days, weeks, or even months.

The profile is typically created with the device 10 in a calibration mode by having the user wear the device 10 while he engages in normal activity that avoids receiving any significant impacts. For example, if the user is playing American football, he may wear the head component 24, such as a football helmet, and the body component 26, such as shoulder pads. The other components of the device 10, such as the first sensor 12, the second sensor 14, and the first and second linkage elements 16A, 16B may be connected as discussed above. The user may play or practice football, kicking or punting the ball, running with the ball, throwing and catching passes, etc., without tackling or being tackled. All the while, the first and second sensors 12, 14 are transmitting measured data on the user's normal, voluntary, non-injurious, and non-concussion-inducing head movement or body movement to the processing element 22. In some instances, the data may be first stored in the memory element 20.

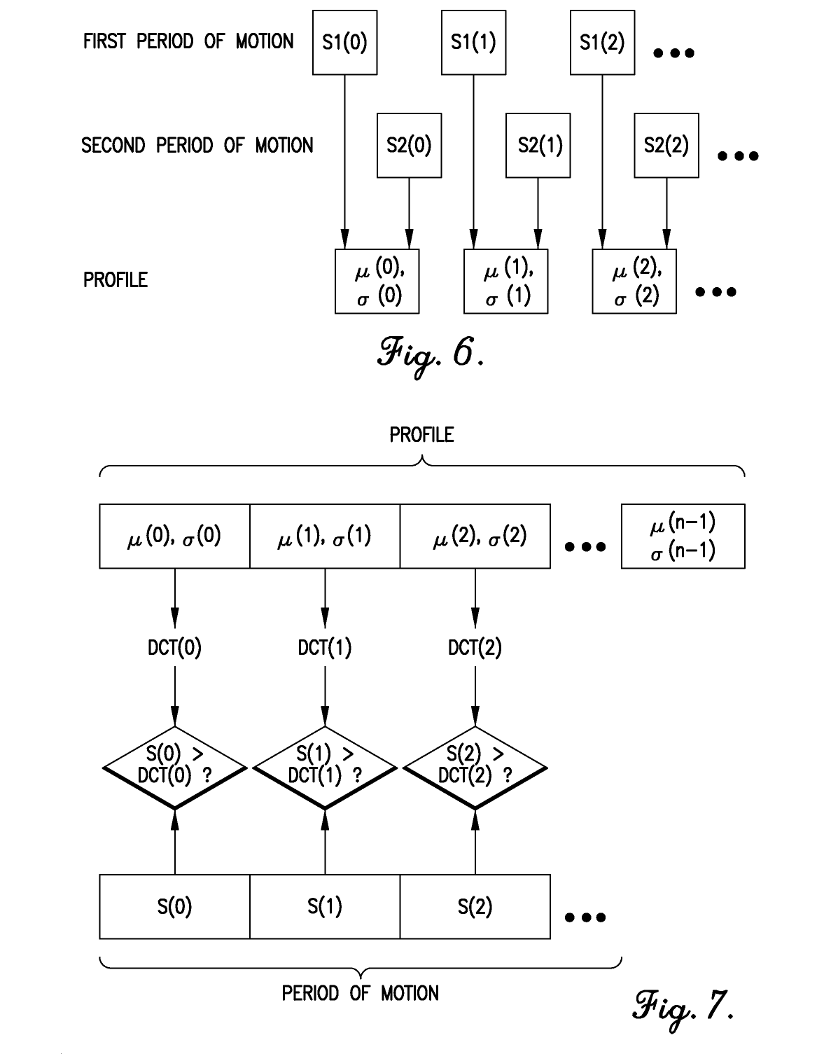

The data may be sampled and generated from the first and second sensors 12, 14 at a given frequency, for example, 1 kiloHertz (kHz), resulting in one real-time measured sample every millisecond (1 ms). The processing element 22 may parse the data to identify a plurality of periods of motion, wherein each period of motion is the time during which the user's head, body, or both are moving. The processing element 22 may determine the point at which the real-time measured samples transition from being less than a motion threshold to greater than the motion threshold, wherein the motion threshold is a value of the measured data which indicates that motion of the user's head or body is occurring. For the first period of motion, the processing element 22 may then indicate that the data received from the first and second sensors 12, 14 after the user starts moving is period of motion data. The indication may include tagging the subsequent data, placing the subsequent data in a specific location in the memory element 20, creating a virtual table or a database for the subsequent data, or the like. The period of motion data may include a stream of real-time measured samples, indicated in FIG. 6 as S1(n), where n may be 0, 1, 2, and so on. In some embodiments, each real-time measured sample may also include a timestamp indicating the time (of day) when the data value was measured. Thus, in an exemplary embodiment, the period of motion data may include or represent the measured value of the acceleration of the user's head or body in the first millisecond, the second millisecond, the third millisecond, and so forth after motion first started. The processing element 22 may continue indicating or marking the data from the first and second sensors 12, 14 as period of motion data until the value of any of the real-time measured samples falls below the motion threshold—indicating that the period of motion has ended.

Afterward, the processing element 22 may parse the data from the first and second sensors 12, 14 until it determines that the next period of motion, indicated in FIG. 6 as S2(n), has begun. Then, in an ongoing and real-time process, for each subsequent period of motion, the processing element 22 may determine the mean p, or average, of the first real-time measured sample of the current period of motion with the first real-time measured sample of all of the previous periods of motion. For example, using the real-time measured samples from just two periods of motion as shown in FIG. 6, the processing element 22 may calculate μ(0) as the mean of S1(0) and S2(0). The processing element 22 may also calculate other statistical data, such as the standard deviation (SD) or the standard error of the mean (SEM), etc. For example, using the real-time measured samples from just a few periods of motion as shown in FIG. 6, the processing element 22 may calculate σ(0) as the standard deviation of S1(0), S2(0), etc. Furthermore, the processing element 22 may perform similar calculations of mean and standard deviation for the second and third and all of the subsequent real-time measured samples of the current period of motion. The statistical calculations of the mean, SD, SEM, and so forth for each sequential real-time measured sample may become a part of the contents of each cell of the profile. Thus, the first cell of the profile may include μ(0), σ(0), and possible other statistical data. The second cell may include at least μ(1) and σ(1). The other cells may follow suit.

Values of movement parameters (velocities, accelerations, etc.) may be statistically analyzed as the mean±SD. The entire profile, including the raw data as well as the results of statistical analysis, may be transmitted to a memory storage via a central server or via a cloud memory storage. The movement parameters (velocities, accelerations, etc.) may be further organized. For example, those movement parameters (velocities, accelerations, etc.) on head movements may be organized into pitch, roll, yaw, or oblique files. Those movement parameters (velocities, accelerations, etc.) on body movements may be organized into similarly mutually orthogonal directional files. If necessary, one-tailed independent Student's t-tests or Mann-Whitney rank sum tests may be used to test differences between two sets of data that passed or failed the normality test, respectively.

The device 10 may be switched from the calibration mode to a normal mode after a certain number, such as 1,000 or more, of periods of motion have been analyzed or after a certain period of time has elapsed, such as one or two months. Alternatively, the mode of the device 10 may be switched from calibration to normal by an administrator or manufacturer via a manual operation of by sending a wireless signal to the communication element 18 when it is deemed that normal, voluntary, non-injurious, and non-concussion-inducing motion has been properly captured in the profile. Once the mode is switched, the profile may include a time-sequence ordered set of mean values, standard deviations, and other statistical values for parameters such as position, velocity, and acceleration for the head and the body.

Once the profile is created, the device 10 may be configured or programmed to operate in the normal mode during which the user is engaging in activity that may involve impacts which could potentially cause a concussion. It is generally accepted that a concussion may occur when acceleration, as an exemplary parameter, of the head is greater than a concussion threshold value. In the present invention, the operation of the processing element 22 does not involve a single, fixed, and one-size-fits-all threshold. Instead, the operation of the processing element 22 is based on a dynamic concussion threshold (DCT), which is unique to each individual user. Therefore, value or values for DCT in units of g (gravity) may vary from user to user, just like the fact that the realistic concussion threshold may vary from user to user. The processing element 22 may also set a different DCT for the same user dependent upon the directionality of the impact (e.g. in the plane of pitch, roll, or yaw for head movements). The value or values of DCT may further vary with a neck stiffness index (see below). In the present invention, value or values for DCT are determined relative to the user's mean, or average, levels of acceleration during normal activity. Here the term “normal activity” is defined as those head or body movements that are normal, voluntary, non-injurious, and non-concussion-inducing. In addition, the determination of DCT should take into account average levels of variation, or standard deviation, of acceleration during normal activity. For example, the processing element 22 may determine the dynamic concussion threshold value to be equal to the mean value of acceleration plus the standard deviation multiplied by a sensitivity factor, the value of which may be determined with considerations on the neck stiffness index.

Because the head is connected to the body via the neck, both the head and the body move whenever a force acts on the head or the body of the user. The relationship between the biomechanical parameters of head movement and those of body movement is determined by the characteristics of the neck. The processing element 22 may further determine or calculate a neck stiffness index as a ratio of the data, such as acceleration, from the second sensor 14 to the data, such as acceleration, from the first sensor 12. For example, the processing element 22 may average a plurality of data samples from the second sensor 14 and divide that value by an average of a plurality of data samples from the first sensor 12. If the user's neck is perfectly stiff or nearly as stiff as a steel column the size of the user's neck, then the output of the first sensor 12 and the second sensor 14 may be generally close. Indeed, if the external force acts on the center of mass of the head-and-neck construct with a perfectly stiff neck, the output of the first sensor 12 and the second sensor 14 may be identical, generating a neck stiffness index of unity or one. Generally, if a force acts on the head with a neck less stiff than perfect may result into a condition in which the first sensor 12 (affixed to the head) may register a larger acceleration than the second sensor 14 (affixed to the body), generating a neck stiffness index smaller than unity. This may be particularly the case when the impact force is delivered to the head. Our preliminary laboratory observation indicated that a male college student with a heavy neck may have 0.6 as his neck stiffness index while a female college student with a slender neck may have 0.4 as her neck stiffness index. The neck stiffness index may also be a function of the position, velocity, and acceleration of head movement and body movement. The neck stiffness index may be stored in the memory element 20 and is unique for each user. Ultimately, information on the neck stiffness index on a given user may also be transmitted to a memory storage via a central server or via a cloud memory storage. The utility of the neck stiffness index is in an application in sub-concussive protection rendered by the processing element 22 via a sensitivity factor (see below).

Since there is a mean and a standard deviation for each sequential value of velocity, acceleration, etc. in the profile, there is a dynamic concussion threshold for each sequential value at each level of the sensitivity factor. For example, if the sensitivity factor is equal to one, then, a head movement that exceeds the DCT is defined as those head movements with head velocity or acceleration that are equal to or more than the mean value plus one standard deviation. Any head movement with biomechanical parameters (e.g. head velocities, accelerations, etc.) exceeding the DCT by one standard deviation has a probability of 15.9% as being part of the user's normal head movement. Alternatively, the processing element 22 may conclude that there is an 84.1% probability that such head movement may be caused by potentially injurious external forces (as opposed to initiated by the user). For another example, if the sensitivity factor is equal to two, then, a head movement that exceeds the DCT is defined as those head movements with head velocity or acceleration that are equal to or more than the mean value plus two standard deviation. Any head movement with biomechanical parameters (e.g. head velocities, accelerations, etc.) exceeding the DCT by two standard deviation has a probability of 2.3% as being part of the user's normal head movement. Alternatively, the processing element 22 may conclude that there is a 97.7% probability that such head movement may be caused by potentially injurious external forces (as opposed to initiated by the user). For a third example, if the sensitivity factor is equal to three, then, a head movement that exceeds the DCT is defined as those head movements with head velocity or acceleration that are equal to or more than the mean value plus three standard deviation. Any head movement with biomechanical parameters (e.g. head velocities, accelerations, etc.) exceeding the DCT by three standard deviation has a probability of 0.1% as being part of the user's normal head movement. Alternatively, the processing element 22 may conclude that there is a 99.9% probability that such head movement may be caused by potentially injurious external forces (as opposed to initiated by the user). The numbers 15.9, 2.3, 0.1% are taken directly from a statistical table on one-tailed normal distribution.

In some embodiments, therefore, it may be possible for the user, or an administrator, to adjust the formula used by the processing element 22 to calculate the dynamic concussion threshold for each sequential value in the profile incorporating the sensitivity factor. For example, the formula may include a sensitivity factor (SF) in the following fashion: DCT(n)=μ(n)+SF×σ(n). Values of the sensitivity factor may be determined with consideration of the neck stiffness index. Generally, a smaller SF value may be used in a user with a neck stiffness index associated with a more flexible or less stiff neck. Also generally, smaller values of the SF render the device 10 more sensitive or restrictive, i.e., the locking signal is asserted at lower values of acceleration. Larger values of the SF render the device 10 less sensitive or restrictive, i.e., the locking signal is asserted at higher values of acceleration. Exemplary values of the SF may range from less than one to three or higher.

The processing element 22 may parse real-time data that is supplied by the first and second sensors 12, 14. Once the start of a period of motion is detected, the processing element 22 may compare the real-time measured samples with the dynamic concussion threshold, as depicted in FIG. 7. The processing element 22 may retrieve the mean μ(0) and the standard deviation σ(0) for the first sequential value in the profile and compute the first value of the dynamic concussion threshold DCT(0), which is then compared to the first real-time measured sample S(0), depicted as S(0)>DCT(0)? The processing element 22 may further compute a second value of the dynamic concussion threshold that is compared with the second real-time measured sample. The processing element 22 may continue computing values of the dynamic concussion threshold and comparing them with the corresponding real-time measured samples for all of the real-time measured samples of the current period of motion.

To summarize the description up to this point, the dynamic concussion threshold (DCT) in the present invention is not a single, one-size-fits-all, quantity. Instead, it is determined based on an individualized profile of the user's own normal, voluntary, non-injurious, and non-concussion-inducing head and body movements. Because the DCT is based on the profile of the user's own normal, non-injurious, and non-concussion-inducing head and body movements, DCT is generally at a lower g force value than a force value that is known to cause or likely to cause a concussion. In addition, the sensitivity factor (SF), determined together with the neck stiffness index can offer the opportunity for adjusting the level of protection. My making the generation of a locking signal at a lower acceleration, the operation of the processing element 22 becomes more sensitive while, at the same time, it may be possible to include protection against sub-concussive impact events.

From published accelerometer data obtained from football players, it is possible that the acceleration possibly leading to a concussion reaches its peak value between 10 ms and 15 ms after impact. Also from published accelerometer data obtained from football players, it is possible that the acceleration possibly leading to a concussion lasts no more than 100 or 200 ms. From high-speed video analysis of boxing matches, it is possible that a concussion process may start or take place between 30-50 ms after an impact of the knockout type. These data dictates that a preventive countermeasure to reduce the risk of concussion must be launched quickly, preferably within single digit of ms and not longer than 30 ms when a concussion may take place. In addition, the duration of the countermeasure needs not to be longer than 100 or 200 ms.

The sensitivity of modern accelerometers is generally of the order of a fraction of 1 G. The peak or maximal force or acceleration involved in a concussion is generally of the order of 30 g or more. The acceleration or force increases to reach a peak or maximum value in a monotonic fashion with time, typically within 20 ms after the impact. For these reasons, it may be possible to detect the trajectory of acceleration that might cause a concussion within a short time period, before the acceleration reaches its peak value—for example, approximately 5 ms to approximately 7 ms after an impact when sensors 12 and 14 may register forces with magnitudes that generally do not cause brain injury or concussion, e.g. between 5-10 g, or less. Thus, the processing element 22 may start a counter or otherwise track the values of the real-time measured samples right after the identification of a period of motion (see FIG. 6) in order to determine the timing and the occurrence of a first real-time measured sample whose value exceeds the dynamic concussion threshold. If the processing element 22 determines that each value of the consecutive real-time measured samples for at least 5-7 ms worth of real-time measured samples exceeds the dynamic concussion threshold, then it is likely that a concussion may occur unless countermeasures are taken. The value of 5-7 ms is selected to describe our invention as exemplary time periods because it was demonstrated in the inventor's laboratory that commercial grade accelerometers can be readily programmed to predict the peak magnitudes of impact forces (which generally occur at 10-20 ms after impact) from data in the first 5-7 ms of impact with a success rate generally better than 99%. Consequently, the processing element 22 may generate the locking signal while the sensors 12 and 14 are still experiencing and reporting accelerations that are significantly under values that could cause brain injury or a concussion. In this way, the locking signal in the present invention is therefore generated ahead of or before a concussion could take place. The locking signal may be embodied by an electrical voltage, an electrical current, or another electrical parameter, or a binary data stream, a binary code, or the like.

The locking signal may be generated, activated, or asserted for a range from approximately 100 ms to approximately 200 ms. Then, the locking signal is deactivated or deasserted. Because the duration of the locking signal is between 100 to 200 ms, the accompanying rigidity of the linkage elements 16A and 16B may only impose minimal effect on the degree of freedom of head movements (an eye blink is generally between 300 to 400 ms). Thus the user's ability to engage in athletic activities such as during a game of American football or to engage in required tasks such as during training or during combat, etc. may only be affected minimally.

To summarize the description up to this point, the onset and the duration of the locking signal issued by the processing element 22 as well as the onset and the duration of the rigidity of the linkage elements 16A and 16B are both fast and brief. The fast onset may be rendered before impact energy can cause a concussion. In addition, the brief rigidity may be rendered as to impose minimal interference to the degree of freedom in head movements.

The processing element 22 may be further configured or programmed to update the profile. When the device 10 is in the normal mode, the processing element 22 continues to identify periods of motion in order to determine whether real-time measured samples of acceleration, for example, may potentially lead to a concussion. At the same time the processing element 22 is comparing the real-time measured samples with the corresponding values of the profile, the processing element 22 may also incorporate the most recently measured real-time samples of acceleration, velocity, etc. into the averaged values of the corresponding profile as long as the most recently measured real-time samples of acceleration, velocity, etc. do not cause the processing element 22 to generate a locking signal. That is, the processing element 22 may incorporate into average the first measured acceleration value for the head of the current, real-time period of motion with the first value of the profile for the same parameter in order to arrive at a new average. The processing element 22 may also calculate a new standard deviation and other statistical values. In this way, the processing element 22 may continuously update the profile by calculating the mean, the standard deviation, and the like for each sequential real-time measured sample and corresponding profile value. In some embodiments, the processing element 22 may not perform these calculations if it determines that the real-time measured sample values are greater than the concussion threshold, because those real-time measured samples are likely indicative of abnormal motion, which should not be included in the profile describing the user's normal, voluntary, non-injurious, and non-concussion-inducing head and body movements. The processing element 22 may replace the values in the profile with the newly calculated mean, standard deviation, and the like for each of the parameters in the profile. In this way, the processing element 22 adapts the profile together with the DCT to the changing behavior of the user over time. The history of such adaption or changes may also be stored in memory (see below).

The processing element 22 may also be configured or programmed to communicate the real-time measured data to a central computer server or a cloud memory and computing mechanism. The processing element 22 may communicate the data to the communication element 18 which in turn, transmits the data wirelessly to the server. Alternatively, the data may be transferred from the first and second sensors 12, 14 to the communication element 18. The server may then execute a software application which receives the real-time measured data from a plurality of devices 10. The software application may perform additional statistical calculations, such as the average or mean, the standard deviation, and the like, on the data. Thus, the software application may create a general population profile. In certain instances, the software application may create population profiles that are age specific, gender specific, and the like. In some embodiments, the device 10 in conjunction with data from the central server or a cloud memory and computing mechanism may further use the age specific, gender specific population profile to identify user or users whose device 10 may be generating data that is sufficiently “out of the norm” to warrant subsequent scrutiny, including a service call in order to identify whether the underlying reason for being “out of the norm” concerns the performance of the equipment or concerns the performance of the user.

In some other embodiments, the device 10 may have the general population profile stored in the memory element 20 when the device 10 is manufactured, thereby eliminating the use of the calibration mode, because the profile would already be built. The processing element 22 may also derive the profile for the individual user over time in the manner described above.

The device 10 may operate as follows. The user may wear the device 10, the body component 26, and the head component 24, as shown in FIGS. 1 and 2. The first sensor 12 may be coupled to the head component 24, and the second sensor 14 may be coupled to the body component 26. The first linkage element 16 may be coupled to the left side of the head component 24 and the upper left side of the body component 26. The second linkage element 16 may be coupled to the right side of the head component 24 and the upper right side of the body component 26. In addition, the communication element 18, the memory element 20, and the processing element 22 may be mounted on a printed circuit board, or similar substrate, that is typically sealed within a package. Furthermore, the package may be attached to the interior of the head component 24 or housed in a cavity of the body component 26.

In some embodiments, the user may initially operate the device 10 in the calibration mode in order to create head movement and body movement profiles individualized to the user. The profile is needed for the processing element 22 to determine what motion of the head and body is normal, voluntary, non-injurious, non-concussion-inducing and what motion of the head and body may lead to a concussion. While the device 10 is in the calibration mode, the processing element 22 does not generate the locking signal and the first and second linkage elements 16A, 16B may always remain flexible. In order to properly create the profile, the user may engage in normal “safe” activity. For example, if the user plays American football, then he may kick or punt the ball, run with the ball, throw and catch passes, etc., without being tackled or tackling. The first and second sensors 12, 14 transmit data to the processing element 22 on a continuous, or near-continuous, basis. The processing element 22 analyzes the data to determine when the user is engaged in periods of motion. During each period of motion, the processing element 22 performs calculations to develop the profile, as indicated in FIG. 6. Once sufficient data is collected to create the profile, the mode of the device 10 can be switched from the calibration mode to the normal mode.

In other embodiments, the device 10 may include a general population profile when it is assembled. Thus, it is not necessary for the user to initially operate the device 10 is the calibration mode.

After the profile is created, or if the device 10 includes a general population profile, the device 10 may operate in the normal mode. The normal mode is for use when the user engages in activity, such as American football, wherein there is the potential for a concussion to occur or there is a need to render sub-concussive to the user. During the activity, the first and second linkage elements 16A, 16B are normally flexible, allowing a full range of motion between the head component 24 and the body component 26. The first and second sensors 12, 14 are continuously, or nearly continuously, transmitting measured data, or real-time measured samples, to the processing element 22. The processing element 22 analyzes the data to determine when the user is engaged in periods of motion. Once a period of motion begins, the processing element 22 may monitor the data, as indicated in FIG. 7, to detect the beginning of motion that may lead to a concussion. Specifically, the processing element 22 may determine whether the real-time measured sample values exceed the dynamic concussion threshold for a time period of approximately 5-7 ms or more, but not longer than approximately 20-30 ms when a concussion may start or take place. If the processing element 22 detects this sequence of potentially injurious motion, then the processing element 22 may generate the locking signal.

The locking signal may be received by the locking elements 38 of the first and second linkage elements 16A, 16B. In exemplary embodiments, each locking element 38 may include a solenoid with a movable core that can be extended into the bypass element 36 of each linkage element 16A, 16B. When the locking element 38 of each linkage element 16A, 16B receives the locking signal, the locking element 38 extends the core into the bypass element 36 of each linkage element 16A, 16B such that the flow of the fluid medium 46 is stopped—rendering each linkage element 16A, 16B rigid. As a result, the relative position of the head component 24 to the body component 26 is locked or frozen. In this locked or frozen state, the rigidity of the linkage element 16A and 16B provides an impedance-preferred pathway such that the energy of the impact to the head is preferentially dissipated through the body component 26 and the user's body, thereby preventing the energy of the impact from causing brain injury. The locking signal may be generated such that the locking element 38 is active and the linkage elements 16A, 16B are rigid for approximately 100 ms to approximately 200 ms. After that time period, the locking signal is no longer generated, the locking element 38 retracts its core from the bypass element 36, the fluid medium 46 may flow, and the linkage elements 16A, 16B are flexible again.

The sensitivity of the device 10 may be adjusted by varying the sensitivity factor which adjusts the value of the dynamic concussion threshold. Generally, decreasing the sensitivity factor renders the device 10 more restrictive, while increasing the sensitivity factor makes the device 10 less restrictive. The sensitivity factor may be adjusted through a software interface, such as a mobile electronic device (smartphone, tablet, or notebook) app. The value of the sensitivity factor may then be transmitted wirelessly from the mobile electronic device to the communication element 18, which in turn transfers the value to the processing element 22 or stores the value in the memory element 20.

While the device 10 is in the normal mode, the profile may be updated while the user is engaged in activity that does not potentially cause a concussion. The processing element 22 may perform the same statistical calculations discussed above for the creation of the profile on head and body movements that are normal, voluntary, non-injurious, and non-concussion-inducing. Thus, the profile adapts as the user becomes more proficient at the sport, gains or loses weight, or otherwise changes his normal pattern of motion. Since the dynamic concussion threshold DCT is computed based on this profile via the formula DCT(n)=μ(n)+SF×σ(n), DCT may also adapt accordingly. This feature may be particularly useful if the device 10 originally includes a general population profile, because over time, the profile may become personalized to the user.

The device 10 may also transmit the real-time measured data from the first and second sensors 12, 14 to a central computer server or a cloud memory or computing mechanism. The server or the cloud may execute a software application which performs additional statistical calculations in order to build a population profile over a plurality of device 10 from a plurality of users. Thus, the profile created by the server may include a general population profile. In certain instances, the software application may create population profiles that are age specific, gender specific, and the like.

Having described the function of device 10, we now describe some additional embodiments.

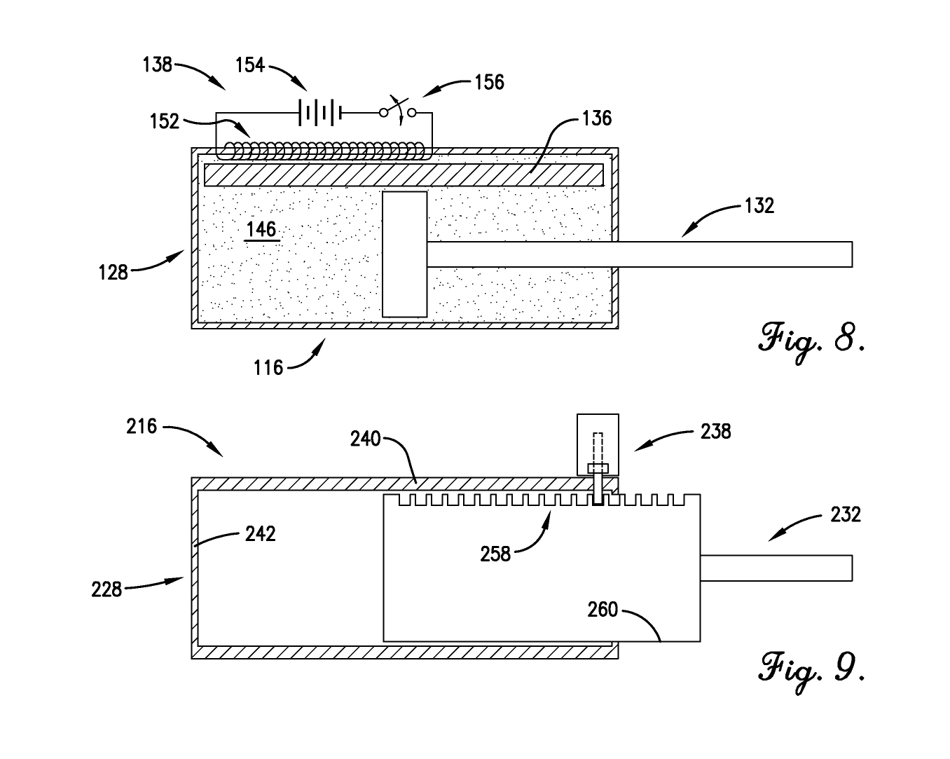

A second embodiment of the first linkage element 116 is shown in FIG. 8 and may include a first member 128, a second member 132, a bypass element 136, a locking element 138, and a fluid medium 146. The first linkage element 116 may further include first and second end joints which are substantially similar in structure and function to the first and second end joints 30, 34, but are not shown in the figures. The first member 128, the second member 132, and the bypass element 136 may also be substantially similar in structure and function to the first member 28, the second member 32, and the bypass element 36, respectively. Furthermore, a second embodiment of the second linkage element, not shown in the figures, may be substantially similar in structure and function to the first linkage element 116.

The locking element 138 may include a coil 152 of electrically conductive material, such as any one of a plurality of metals known in the art, that is wound around at least a portion of the bypass element 136. The coil 152 is electrically connected to an electric power source 154, such as an electric voltage supply or an electric current supply. In addition, the locking element 138 may include a switching element 156, that when switched to an open state prevents electric current flow to the coil 152, and when switched to a closed state allows electric current flow to the coil 152. When the switching element 156 is in the closed state and electric current is flowing through the coil 152, the coil 152 may generate a magnetic field. Furthermore, the switching element 156 may receive the locking signal from the processing element 22, wherein the locking signal is operable to change the state of the switching element 156.

The fluid medium 146 may include, or be embodied by, a magnetorheological material whose viscosity may be adjusted, or varied, by magnetic field. In a first state, in the absence of a magnetic field, the magnetorheological material may be free flowing and may have a relatively low viscosity. In a second state, in the presence of a magnetic field, the magnetorheological material may have a significantly higher viscosity causing the fluid medium 146 to stop, or nearly stop, flowing.

The first linkage element 116 may operate as follows. When the user is active and experiencing normal, non-concussion-inducing motion, the processing element 22 does not generate the locking signal, and thus, the switching element 156 of the locking element 138 is in the open state, resulting in free flow of the fluid medium 146 and flexible motion of the first linkage element 116. When the user experiences motion from an impact that may potentially cause a concussion, the processing element 22 may detect that the acceleration, for example, is greater than the dynamic concussion threshold for a first period of time and may generate the locking signal. The locking element 138 may receive the locking signal which closes the switching element 156, allowing current flow through the coil 152 which generates a magnetic field and increases the viscosity of the fluid medium 146. Generally, when the processing element generates a locking signal, the viscosity of the magnetorheological material may increase significantly within single digit of ms. At a relatively higher viscosity, the fluid medium 146 stops, or nearly stops, flowing—thereby rendering the first linkage element 116 rigid and locking its position at the approximate time when the locking signal was generated. The processing element 22 may stop generating the locking signal after a short period of time, for example approximately 100 ms to approximately 200 ms. In the absence of the locking signal, the fluid medium 146 no longer receives the magnetic field from the coil 152 and may flow freely—thereby rendering the first linkage element 116 flexible again.

A third embodiment of the first linkage element 216 is shown in FIG. 9 and may include a first member 228, a second member 232, and a locking element 238. The first linkage element 216 may not include a bypass element or a fluid medium. A third embodiment of the second linkage element, not shown in the figures, may be substantially similar in structure and function to the first linkage element 216.

The first member 228 may include a sidewall 240 and a first end wall 242, similar in structure and function to the sidewall 40 and the first end wall 42. The first member 228 may not include a second end wall. The second member 232 may include a rod 248 and a disc 250 with an elongated cylindrical shape and having a plurality or recesses 258, or impressions, distributed axially on a sidewall 260 of the disc 250. Each recess 258 may be oriented in a circumferential direction on the sidewall 260. The locking element 238 may include a solenoid that is coupled to the sidewall 240 of the first member 228. The solenoid may include a movable core or plunger that can be extended into an opening in the sidewall 240 and inserted into one of the recesses 258 on the sidewall 260. The extension of the core may be controlled by the locking signal from the processing element 22.

The first linkage element 216 may operate as follows. When the user is active and experiencing normal, non-concussion-inducing motion, the processing element 22 does not generate the locking signal, and accordingly, the core of locking element 238 solenoid is not extended. The second member 232 of the first linkage element 216 moves freely in a telescoping fashion in and out of the first member 228. When the user experiences motion from an impact that may potentially cause a concussion, the processing element 22 may detect that the acceleration, for example, is greater than the dynamic concussion threshold for a first period of time and may generate the locking signal. The locking element 238 may receive the locking signal and the solenoid may extend its core into one of the recesses of the second member 232, thereby preventing any motion of the second member 232 relative to the first member 228 and rendering the first linkage element 216 rigid. The processing element 22 may stop generating the locking signal after a short period of time, for example approximately 100 ms to approximately 200 ms. In the absence of the locking signal, the core of the locking element 238 solenoid may retract from one of the recesses of the second member 232—thereby allowing free motion of the second member 232 and rendering the first linkage element 216 flexible again.

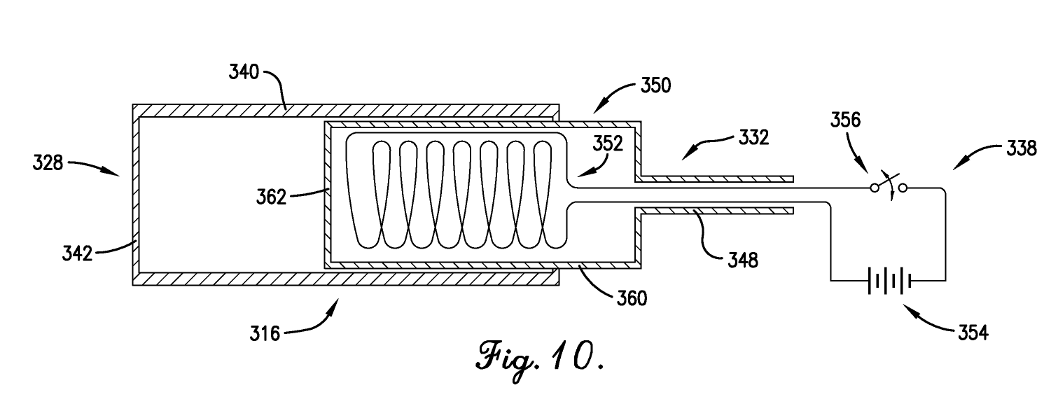

A fourth embodiment of the first linkage element 316 is shown in FIG. 10 and may include a first member 328, a second member 332, and a locking element 338. A fourth embodiment of the second linkage element, not shown in the figures, may be substantially similar in structure and function to the first linkage element 316.

The first member 328 may include a sidewall 340 and a first end wall 342, similar in structure and function to the first end wall 42 and the sidewall 40, but excluding an end wall. The sidewall 340 may be formed from magnetic material such as iron or steel. The second member 332 may include a rod 348 and a disc 350 with a hollow elongated cylindrical shape and having a sidewall 360 and a first end wall 362. The sidewall 360 may be formed from magnetic material such as iron or steel.

The locking element 338 may include a coil 352, an electric power source 354, and a switching element 356. The coil 352 may be formed from electrically conductive material and may be positioned such that the outer edge of the coil rings is adjacent to an inner surface of the sidewall 360 of the second member 332. The electric power source 354 may include, or be embodied by, an electric voltage supply or an electric current supply. The coil 352 may be electrically connected to the electric power source 354 through the switching element 356, that when switched to an open state prevents electric current flow to the coil 352, and when switched to a closed state allows electric current flow to the coil 352.

The first linkage element 316 may operate as follows. When the user is active and experiencing normal, non-concussion-inducing motion, the processing element 22 does not generate the locking signal, and thus, the locking element 338 is not engaged so that the second member 332 of the first linkage element 316 moves freely in a telescoping fashion in and out of the first member 328. When the user experiences motion from an impact that may potentially cause a concussion, the processing element 22 may detect that the acceleration, for example, is greater than the dynamic concussion threshold for a first period of time and may generate the locking signal. The locking element 338 may receive the locking signal which closes the switching element 356, allowing current flow through the coil 352 which generates a magnetic field and a force of attraction. The attractive force from the coil 352 attracts electromagnetically the sidewall 360 of the second member 332 and the sidewall 340 of the first member 328, which generally stops the relative motion of the two members 328, 332 and renders the first linkage element 316 rigid. The processing element 22 may stop generating the locking signal after a short period of time, for example, approximately 100 ms to approximately 200 ms. In the absence of the locking signal, the coil 352 no longer generates the attractive force, which allows the first and second members 328, 332 to move freely and renders the first linkage element 316 flexible again.

Having described a number of additional embodiments, we now describe some relevant processes of the device 10.

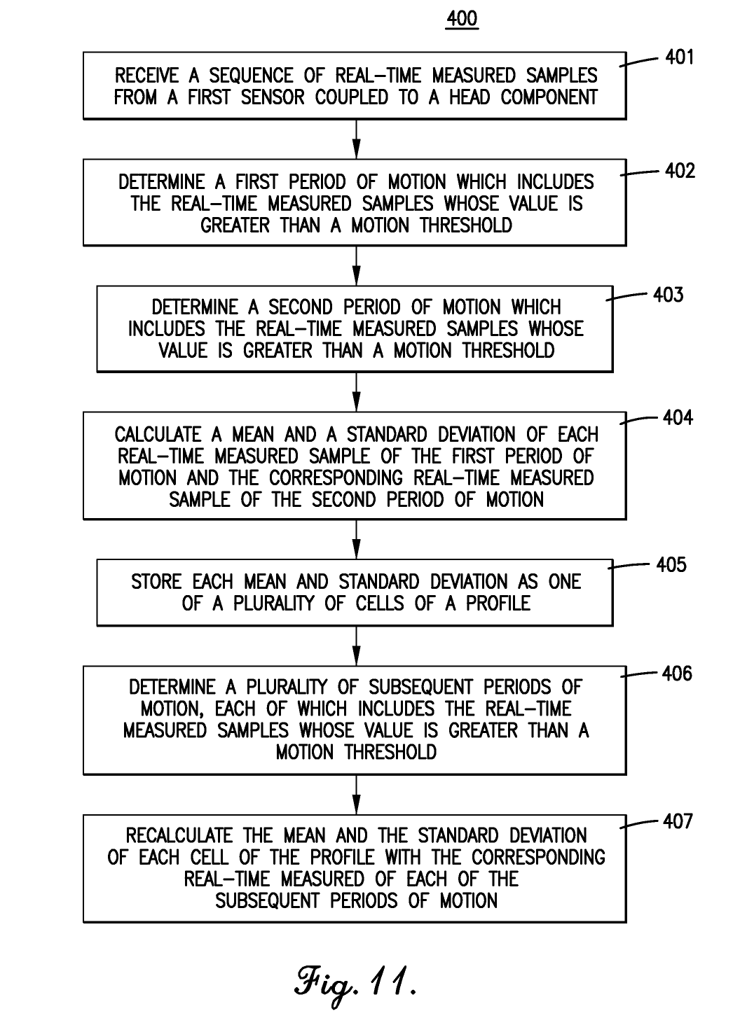

At least a portion of the steps of a method 400 for creating a profile to be used with a device for reducing traumatic brain injury, in accordance with another embodiment of the current invention, is shown in FIG. 11. The steps may be performed in the order presented in FIG. 11, or they may be performed in a different order. In addition, some of the steps may be performed simultaneously instead of sequentially. Furthermore, some steps may not be performed. At least a portion of the steps listed are performed by a processing element 22 of the device 10.

Referring to step 401, a sequence of real-time measured samples is received from a first sensor 12 coupled to a head component 24. The first sensor 12 may include one or more components capable of measuring one or more parameters, such as acceleration, along one or more axes or directions, such as pitch, roll, and yaw. The head component 24 may be worn on a user's head and may include or be embodied by an American football helmet. The first sensor 12 may be positioned in the interior of the helmet and may generate the real-time measured samples at an exemplary frequency ranging from 500 hertz (Hz) to 20 kilohertz (kHz) or higher. The second sensor 14 and the body component 26 may be substantially similar.

Referring to step 402, a first period of motion, as indicated in FIG. 6, is determined, wherein the first period of motion includes the real-time measured samples, S1(n), whose value is greater than a motion threshold. The value of each real-time measured sample may be compared, using the processing element 22, to the motion threshold, a constant whose value indicates that the user is moving. The first sample of the stream of real-time measured samples that is greater than the motion threshold becomes the first sample of the first period of motion. The subsequent real-time measured samples whose values are greater than the motion threshold may also be included in the first period of motion. The last real-time measured sample whose value is greater than the motion threshold is the last sample of the first period of motion.

Referring to step 403, a second period of motion, as indicated in FIG. 6, is determined, wherein the second period of motion occurs in time after the first period of motion and includes the real-time measured samples, S2(n), whose value is greater than the motion threshold.

Referring to step 404, a mean, p, and a standard deviation, a, are calculated for each real-time measured sample of the first period of motion and the corresponding real-time measured sample of the second period of motion. For example, referring to FIG. 6, the processing element 22 may calculate μ(0) as the mean of S1(0), S2(0), etc. and σ(0) as the standard deviation of S1(0), S2(0), etc. The subsequent values of p and a are calculated in the same fashion using the subsequent values of S1 and S2.

Referring to step 405, the mean, μ(n), and standard deviation, σ(n), are stored as one of a plurality of cells of a profile. For example, as shown in FIG. 6, the first cell of the profile includes μ(0) and σ(0), the second cell of the profile includes μ(1) and σ(1), and so forth.

Referring to step 406, a plurality of subsequent periods of motion are determined. Each subsequent period of motion is determined in the same fashion as described above. The subsequent periods of motion may occur during one or more active outings of the user.

Referring to step 407, the mean, μ(n), and standard deviation, σ(n), for each cell of the profile are recalculated using the corresponding real-time measured sample of the each of the subsequent periods of motion. For example, during a current period of motion, new values of μ(0) and σ(0) are calculated, by the processing element 22, using S(0) from the current period of motion, such that μ and σ represent the mean and standard deviation, respectively, of the first real-time measured samples of all the periods of motion that have occurred up to that point. The calculations are repeated for all of the other cells of the profile. The creation of the profile may be complete after a certain number of periods of motion, such as 1,000, have been experienced by the user, or a certain amount of time, such as one or two months, have elapsed.

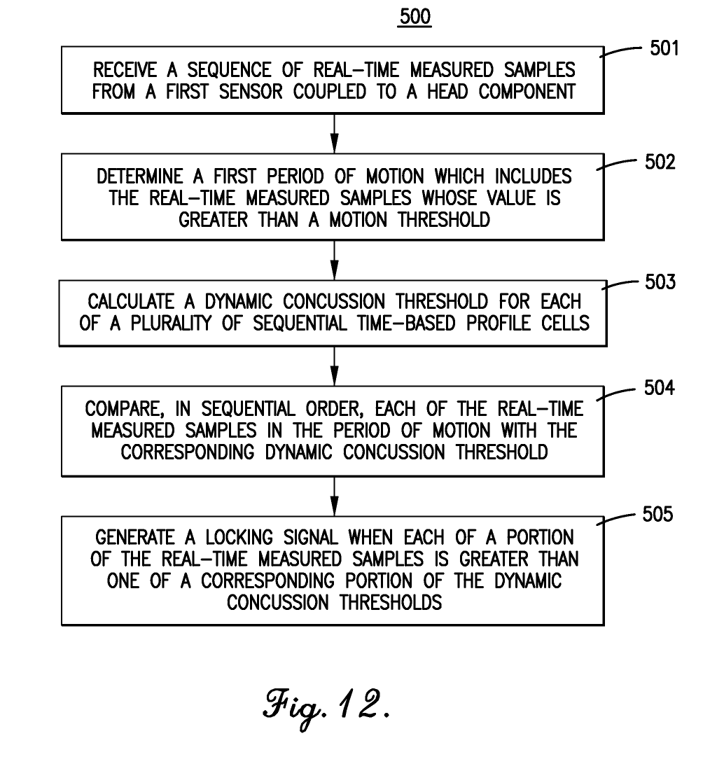

At least a portion of the steps of a method 500 for reducing traumatic brain injury, in accordance with yet another embodiment of the current invention, is shown in FIG. 12. The steps may be performed in the order presented in FIG. 12, or they may be performed in a different order. In addition, some of the steps may be performed simultaneously instead of sequentially. Furthermore, some steps may not be performed. At least a portion of the steps listed are performed by a processing element 22 of the device 10.

Referring to step 501, a sequence of real-time measured samples is received from a first sensor 12 coupled to a head component 24. The first sensor 12 may include one or more components capable of measuring one or more parameters, such as acceleration, along one or more axes or directions, such as pitch, roll, and yaw. The head component 24 may be worn on a user's head and may include or be embodied by an American football helmet. The first sensor 12 may be positioned in the interior of the helmet and may generate the real-time measured samples at an exemplary frequency ranging from 500 hertz (Hz) to 20 kilohertz (kHz) or higher. The second sensor 14 and the body component 26 may be substantially similar.

Referring to step 502, a period of motion, as indicated in FIG. 7, is determined, wherein the period of motion includes the real-time measured samples whose value is greater than a motion threshold. The value of each real-time measured sample may be compared, using the processing element 22, to the motion threshold, a constant whose value indicates that the user is moving. The first sample of the stream of real-time measured samples that is greater than the motion threshold becomes the first sample, S(0), of the period of motion. The subsequent real-time measured samples whose values are greater than the motion threshold may also be included in the period of motion. The last real-time measured sample whose value is greater than the motion threshold is the last sample of the period of motion.

Referring to step 503, a plurality of dynamic concussion thresholds is calculated, one for each of a plurality of sequential time-based profile cells. The profile, as indicated in FIG. 7, generally defines, or quantifies, motion by the user that will likely not lead to a concussion and comprises a plurality of cells, each cell including statistical calculations of real-time measured samples from previous periods of motion. Exemplary statistical calculations include a mean, p, and a standard deviation, a, for each cell. In addition, the order of the cells in the profile corresponds to the order of the real-time measured samples in each of the periods of motion used to develop the profile. Each dynamic concussion threshold, DCT, is calculated as, for example, a sum of the mean and the standard deviation for one of the cells of the profile.

Referring to step 504, each of the real-time measured samples in the period of motion is compared, in sequential order, with the corresponding dynamic concussion threshold. The processing element 22 may compare the first real-time measured sample of the period of motion with the dynamic concussion threshold calculated using the data from the first cell of the profile. The processing element 22 may then compare all the subsequent real-time measured samples with the dynamic concussion thresholds calculated using the data of the subsequent cells. This process is indicated in FIG. 7 as a sequence of decision blocks including: S(0)>DCT(0)?; S(1)>DCT(1)?; S(2)>DCT(2)?; etc.

Referring to step 505, a locking signal is generated when each of a portion of the real-time measured samples is greater than one of a corresponding portion of the dynamic concussion thresholds. The processing element 22 may compare each real-time measured sample of the period of motion with the dynamic concussion threshold calculated from the corresponding profile cell in sequential order. If the processing element 22 determines that a certain number of consecutive samples (representing a first period of time) are greater than the corresponding dynamic concussion thresholds, then the real-time measured samples may indicate the beginning of an impact that could lead to a concussion. It may be that approximately 5-7 ms worth of real-time measured samples that are greater than their corresponding dynamic concussion thresholds is sufficient to mark the beginning of a concussion-causing impact. It may be that more than 5-7 ms worth of real-time measured samples is required. The processing element 22, however, does not wait any longer than 20-30 ms to generate the locking signal so that the linkage elements 16A and 16B can assume their rigidity before a concussion may start or take place. The locking signal may be embodied by an electrical voltage, an electrical current, or another electrical parameter, or a binary data stream, a binary code, or the like. The locking signal may be generated, activated, or asserted for a range from approximately 100 ms to approximately 200 ms. Then, the locking signal is deactivated or deasserted.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A device for reducing traumatic brain injury, the device comprising:
a first sensor coupled to a head component configured to measure an acceleration of a user's head as a result of motion of the head component and to generate a sequence of real-time measured samples;
a first linkage element configured to connect the head component to a body component, the first linkage element switchable between a first state in which it is relatively flexible and a second state in which it is relatively rigid based upon a locking signal; and
a processing element electronically coupled to a memory element, the processing element configured to receive the real-time measured samples and to generate the locking signal when each of a portion of the real-time measured samples is greater than one of a corresponding portion of a plurality of dynamic concussion thresholds.

2. The device of claim 1, wherein the processing element is further configured to determine a plurality of periods of motion, wherein each period of motion is a time period during which the value of each real-time measured sample is greater than a motion threshold.

3. The device of claim 2, wherein each cell includes a mean and a standard deviation of historically collected data for the compilation of a profile for the normal, voluntary, non-injurious, and non-concussing head and body movements, and the processing element is further configured to calculate the dynamic concussion threshold for each profile cell as a sum of the mean and the standard deviation.

4. The device of claim 2, wherein each cell includes a mean and a standard deviation of historically collected data, and the processing element is further configured to calculate the dynamic concussion threshold for each profile cell as a sum of the mean and a product of the standard deviation and a sensitivity factor, wherein the sensitivity factor is an adjustable value which determines the sensitivity of the device to an impact received by the user.

5. The device of claim 1, wherein the processing element is further configured to calculate a dynamic concussion threshold based on a profile for the normal, voluntary, non-injurious, and non-concussing head and body movements compiled over a plurality of periods of motion comprising of a plurality of sequential time-based profile cells, wherein each cell includes a plurality of statistical values of historically collected data from the first sensor.

6. The device of claim 1, wherein the processing element is further configured to generate the locking signal when each of the real-time measured samples generated during a first time period is greater than a corresponding one of the dynamic concussion thresholds.

7. The device of claim 6, wherein the first time period is approximately in the range between 5-7 milliseconds and 20 milliseconds immediately after an impact or is shorter than the time period between the instant of impact and a time when concussion may take place.

8. The device of claim 1, wherein the processing element is further configured to
determine a period of motion which includes the real-time measured samples whose value is above a motion threshold, and
compare, in sequential order, each of the real-time measured samples in the period of motion with the corresponding dynamic concussion threshold.

9. A device for reducing traumatic brain injury, the device comprising:
a first sensor coupled to a head component configured to measure an acceleration of a user's head as a result of motion of the head component and to generate a first sequence of real-time measured samples;
a first linkage element configured to connect the head component to a body component, the first linkage element switchable between a first state in which it is relatively flexible and a second state in which it is relatively rigid based upon a locking signal; and
a processing element electronically coupled to a memory element, the processing element configured to receive the real-time measured samples,
determine a period of motion which includes the real-time measured samples whose value is greater than a motion threshold,
determine a plurality of sequential dynamic concussion thresholds based on a profile for the normal, voluntary, non-injurious, and non-concussing head and body movements compiled over a plurality of periods of motion, and
compare, in sequential order, each of the real-time measured samples in the period of motion with the corresponding dynamic concussion thresholds.

10. The device of claim 9, wherein the processing element is further configured to determine each dynamic concussion threshold as a sum of a mean and a standard deviation from one of a plurality of profile cells, wherein the mean and standard deviation are calculated from data historically collected from the first sensor while the user executes normal, non-injurious, and non-concussion-inducing head or body movements.

11. The device of claim 9, wherein the processing element is further configured to determine each dynamic concussion threshold as a sum of a mean and a standard deviation multiplied by a sensitivity factor from one of a plurality of profile cells, wherein the mean and standard deviation are calculated from real-time measured samples of previously occurring periods of motion.

12. The device of claim 9, wherein the processing element is further configured to generate the locking signal when each of a portion of the real-time measured samples is greater than one of a corresponding portion of the dynamic concussion thresholds.

13. The device of claim 9, wherein the processing element is further configured to generate the locking signal when each of the real-time measured samples generated during a first time period is greater than a corresponding one of the dynamic concussion thresholds.

14. The device of claim 13, wherein the first time period is approximately in the range between 5-7 milliseconds and 20 milliseconds immediately after an impact and is shorter than the time period between the instant of impact and a time when concussion may take place.

15. The device of claim 13, wherein the processing element receives the real-time measured samples and generates the locking signal before an impact force or energy is manifested at the user's head to such an extent that the manifested force or energy may cause brain injury or a concussion.

16. The device of claim 9, wherein the first linkage element includes a tubular chamber retaining hydraulic fluid and a plunger configured to telescopically move within the chamber, the first member coupled to the body component and the second member coupled to the head component.

17. The device of claim 9, wherein the first linkage element includes a tubular chamber retaining pneumatic gas and a plunger configured to telescopically move within the chamber, the first member coupled to the body component and the second member coupled to the head component.

18. The device of claim 9, wherein the first sensor includes a micro electro-mechanical systems (MEMS) device.

19. The device of claim 9, wherein when the first linkage element is in the second state, an impedance-preferred pathway for energy dissipation to the body is established.

20. The device of claim 9, wherein the first linkage element is in the second state for a time period ranging from approximately 100 milliseconds to approximately 200 milliseconds.

21. The device of claim 9, wherein the first sensor is configured to measure acceleration along the pitch, roll, and yaw axes.

22. The device of claim 9, further comprising a second sensor positioned on the body component and configured to measure an acceleration of the user's body and to generate a second sequence of real-time measured samples.

23. The device of claim 22, wherein the first sensor is configured to measure acceleration of the user's head relative to the user's body.

24. The device of claim 22, wherein the processing element is further configured to:
receive the real-time measured samples from the second sensor, and
calculate a neck stiffness index as an average of real-time measured samples from the second sensor divided by real-time measured samples from the first sensor.

25. A method for reducing traumatic brain injury, the method comprising the steps of:
receiving a sequence of real-time measured samples from a first sensor coupled to a head component;
determining a period of motion which includes the real-time measured samples whose value is greater than a motion threshold;
calculating a dynamic concussion threshold for each of a plurality of sequential time-based profile cells; and
comparing, in sequential order, each of the real-time measured samples in the period of motion with the corresponding dynamic concussion threshold.

26. The method of claim 25, further comprising the step of generating a locking signal when each of a portion of the real-time measured samples is greater than one of a corresponding portion of the dynamic concussion thresholds.

27. The method of claim 25, further comprising the step of generating a locking signal when each of the real-time measured samples generated during a first time period is greater than a corresponding one of the dynamic concussion thresholds.

28. The method of claim 27, wherein the first time period is approximately in the range between 5-7 milliseconds and 20 milliseconds immediately after an impact and is shorter than the time period between the instant of impact and a time when concussion may take place.

29. The method of claim 25, wherein each profile cell includes a mean and a standard deviation and the dynamic concussion threshold is calculated as a sum of the mean and the standard deviation.

30. The method of claim 25, wherein each profile cell includes a mean and a standard deviation and the dynamic concussion threshold is calculated as a sum of the mean and a product of the standard deviation and a sensitivity factor, the sensitivity factor being an adjustable value which determines the sensitivity of the device to an impact received by the user.
